(19) United States
(12) Patent Application Publication
(10) Pub. No.: US 2017/0267753 A1
(43) Pub. Date: Sep. 21, 2017

(54) COMBINATION THERAPY FOR CO-ADMINISTRATION OF MONOCLONAL ANTIBODIES

(71) Applicant: Eli D Ehrenpreis, Skokie, IL (US)

(72) Inventor: Eli D Ehrenpreis, Skokie, IL (US)

(21) Appl. No.: 15/605,212

(22) Filed: May 25, 2017

Related U.S. Application Data
(63) Continuation-in-part of application No. 14/947,193, filed on Nov. 20, 2015.
(60) Provisional application No. 62/082,682, filed on Nov. 21, 2014.

(51) Int. Cl. C07K 16/24; A61K 31/4706; A61K 31/165; A61K 39/395

(52) U.S. Cl. CPC C07K 16/241; A61K 39/3955; A61K 31/4706; A61K 31/165; C07K 2317/21; C07K 2317/24; A61K 2039/505

(57) ABSTRACT

Disclosed are methods for enhancing the efficacy of monoclonal antibody therapy, which entails co-administering a therapeutic monoclonal antibody, or a functional fragment thereof, and an effective amount of colchicine or hydroxychloroquine, or a combination thereof, to a patient in need thereof. Also disclosed are methods of prolonging or increasing the time a monoclonal antibody remains in the circulation of a patient, which entails co-administering a therapeutic monoclonal antibody, or a functional fragment of the monoclonal antibody, and an effective amount of colchicine or hydroxychloroquine, or a combination thereof, to a patient in need thereof, wherein the time the monoclonal antibody remains in the circulation (e.g., blood serum) of the patient is increased relative to the same regimen of administration of the monoclonal antibody but without the co-administration of the effective amount of colchicine and/or hydroxychloroquine. Further disclosed are therapeutic combinations, and kits containing the monoclonal antibodies and hydroxychloroquine and/or colchicine.

### CROSS-REFERENCE TO RELATED APPLICATIONS

This application is a continuation-in-part of U.S. patent application Ser. No. 14/947,193, filed Nov. 20, 2015, which claims the benefit of U.S. Provisional Application No. 62/082,692, filed Nov. 21, 2014. The disclosures of each of these are incorporated herein by reference in their entireties for all purposes.

### BACKGROUND

Monoclonal antibodies have been developed for the treatment of a variety of conditions including autoimmune disorders, cancer, asthma, hypercholesterolemia and sepsis. The development of therapeutic monoclonal antibodies in these areas of medicine has progressed at a rapid pace. The number of new biologic agents that have been approved by the Food and Drug Administration (FDA) each year has quadrupled between 2004 and 2008. This relatively new class of medications has resulted in marked improvement in a number of patients with complex and potentially life-threatening conditions, and in some cases, they have replaced traditional small molecule pharmaceuticals as treatments of choice. These medications also accounted for approximately 17% of total global spending on medicines in 2016 with an overall market value of $200 billion.

An important influence on the utility of biologic therapy in an individual patient is the development of anti-drug antibodies (ADA). ADAs have been documented in patients receiving multiple doses of a variety monoclonal antibodies, including infliximab (IFX), a treatment for inflammatory bowel disease, rheumatoid arthritis, psoriatic arthritis and other autoimmune diseases. The development of ADAs to IFX, as well as other monoclonal antibodies, is associated with systemic reactions that can occur during or within a few days of drug infusion. When severe, they can require discontinuation of biologic therapies. In addition, a number of studies have shown that ADAs reduce the efficacy of biologic therapy. From a pharmacokinetic standpoint, ADAs have been shown to enhance the clearance of biologic medications. Strategies that have been developed to prevent ADA formation and their negative effect on the efficacy of biologic therapies include adherence to consistent timing of drug infusions or subcutaneous injection regimens, and the co-administration of oral immunomodulating medications such as thiopurines (azathioprine or its precursor agent 6-mercaptopurine), and methotrexate. Although studies have shown that this strategy of co-administration of the aforementioned immunomodulating agents reduces ADA production, rapid clearance of biologic agents and infusion reactions, patients on both classes of drugs may become further immunosuppressed, placing them at increased risk for opportunistic infections, tuberculosis, overwhelming fungal infections and a variety of cancers. In fact, hepatosplenic T-cell lymphoma, a rare, deadly cancer seen primarily in young males with Crohn's disease, has only been described in patients receiving monoclonal antibodies (MAb) against tumor necrosis factor alpha (TNF-α) in combination with thiopurine drugs.

It is therefore of high importance to develop methods to prevent ADA development in patients receiving biologic therapy that are safer than those that are currently in practice.

### SUMMARY

Broadly, the present invention is based on the unexpected discovery that colchicine and hydroxychloroquine increase the time that a monoclonal antibody remains in the circulation or circulatory system (e.g., blood serum) of a patient by reducing the clearance of the monoclonal antibody from the body. The co-administration of colchicine and hydroxychloroquine and a monoclonal antibody thus increase the clearance time of monoclonal antibody from the body. Unlike known and conventional attempts to increase the effectiveness of monoclonal antibody therapies by preventing the formation of HACAs and other anti-drug antibodies (such as immunosuppression), the present invention may mitigate or even eliminate one of more of these drawbacks.

Accordingly, a first aspect of the present invention is directed to a method for enhancing the efficacy of monoclonal antibody therapy, which entails co-administering a therapeutic monoclonal antibody, or a functional fragment thereof, and an effective amount of colchicine or hydroxychloroquine, or a combination thereof, to a patient in need thereof.

A related aspect of the present invention is directed to a method of prolonging or increasing the time a therapeutic monoclonal antibody remains in the circulation or circulatory system of a patient, which entails co-administering an effective amount of a therapeutic monoclonal antibody, or a functional fragment thereof, and an effective amount of colchicine, hydroxychloroquine, or a combination thereof, to a patient in need thereof. Clearance time of the monoclonal antibody from the circulation (e.g., blood serum) of the patient is increased relative to the same regimen of administration of the monoclonal antibody but without the co-administration of the effective amount of colchicine and/or hydroxychloroquine.

Another aspect of the present invention is directed to a therapeutic combination, which includes a therapeutically effective amount of a monoclonal antibody, and an effective amount of colchicine or hydroxychloroquine, or a combination thereof.

A further aspect of the present invention is directed to a kit, which includes a therapeutic combination, which includes a therapeutically effective amount of a monoclonal antibody, and an effective amount of colchicine or hydroxychloroquine, or a combination thereof. The kit may include both agents in a single dosage form or in separate dosage forms, in which case the respective dosage forms may be disposed in separate containers in the kit. The kit may further include printed instructions for using the therapeutic combination to practice the methods described herein.

In some embodiments of these aspects of the present invention, the monoclonal antibody is chronically administered (over a prolonged period of time) such as in the case of treatment of auto-immune diseases, e.g., monoclonal antibodies that target (and thus inhibit) TNF-α, such as adalimumab, certolizumab pegol, golimumab, and infliximab.

Hydroxychloroquine has a long history of use as an anti-malarial drug. Clinical studies have shown that hydroxychloroquine is not effective as a treatment for IBD, cancer, Clostridium infection, sepsis (except due to malaria), asthma or hyperchloresterolemia. Hydroxychloroquine is used as a disease-modifying anti-rheumatic drug in the treatment of rheumatoid arthritis and is commonly employed as treatment for systemic lupus erythematosis. Thus, hydroxychloroquine is effective treatment for several disorders. However, its specific use as combination therapy to enhance the efficacy of monoclonal antibody therapy has not been investigated. Without wishing to be bound to any particular theory, it is believed that hydroxychloroquine unexpectedly increases the time that a monoclonal antibody remains in the blood serum of a patient in one or more ways. It may decrease the clearance of the monoclonal antibody from the patient's system, for example, by inhibiting or reducing formation of antibodies such as human anti-chimeric antibodies (HACAs) or other anti-drug antibodies and decreasing the removal of the monoclonal antibody from the circulation. It is also believed that hydroxychloroquine raises lysosomal pH, which causes disruption of lysososmal function such as processing of antigens (such as monoclonal proteins) and antigen presentation to mononuclear cells.

### DETAILED DESCRIPTION

Monoclonal antibodies (MAbs) that may be suitable for use in the present invention include human, humanized, chimeric and murine antibodies alike, as well as functional fragments thereof that bind the intended target, e.g., Fab fragments and Scfv fragments, and conjugated (e.g., pegylated MAbs and antibody-drug conjugates) and non-conjugated forms thereof. Representative examples of monoclonal antibodies are set forth in the table below, which FDA-approved MAbs.

TABLE 1
FDA Approved Monoclonal Antibodies

| Antibody | Route | Type | Target | Indication |
|---|---|---|---|---|
| abciximab | intravenous | chimeric Fab | GPIIb/IIIa | Percutaneous coronary |
| | | | | intervention |
| adalimumab | subcutaneous | fully human | TNF | Rheumatoid arthritis |
| adalimumab- | subcutaneous | fully | TNF | Rheumatoid arthritis |
| atto | | human, biosimilar | | Juvenile idiopathic arthritis |
| | | | | Psoriatic arthritis |
| | | | | Ankylosing spondylitis |
| | | | | Crohn's disease |
| | | | | Ulcerative colitis |
| | | | | Plaque psoriasis |
| ado- | intravenous | humanized, antibody- | HER2 | Metastatic breast cancer |
| trastuzumab | | drug conjugate | | |
| emtansine | | | | |
| alemtuzumab | intravenous | humanized | CD52 | B-cell chronic lymphocytic |
| | | | | leukemia |
| alirocumab | subcutaneous | fully human | PCSK9 | Heterozygous familial |
| | | | | hypercholesterolemia |
| | | | | Refractory hypercholesterolemia |
| atezolizumab | intravenous | humanized | PD-L1 | Urothelial carcinoma |
| atezolizumab | intravenous | humanized | PD-L1 | Urothelial carcinoma |
| | | | | Metastatic non-small cell lung |
| | | | | cancer |
| avelumab | intravenous | fully human | PD-L1 | Metastatic Merkel cell |
| | | | | carcinoma |
| basiliximab | intravenous | chimeric | IL2RA | Prophylaxis of acute organ |
| | | | | rejection in renal transplant |
| belimumab | intravenous | fully human | BLyS | Systemic lupus erythematosus |
| bevacizumab | intravenous | humanized | VEGF | Metastatic colorectal cancer |
| bezlotoxumab | intravenous | fully human | Clostridium | Prevent recurrence |
| | | | difficile toxin B | of Clostridium difficile infection |
| blinatumomab | intravenous | mouse, bispecific | CD19 | Precursor B-cell acute |
| | | | | lymphoblastic leukemia |
| brentuximab | intravenous | chimeric, antibody- | CD30 | Hodgkin lymphoma |
| vedotin | | drug conjugate | | Anaplastic large-cell lymphoma |
| brodalumab | subcutaneous | chimeric | IL17RA | Plaque psoriasis |
| canakinumab | subcutaneous | fully human | IL1B | Cryopyrin-associated periodic |
| | | | | syndrome |
| capromab | intravenous | murine, radiolabeled | PSMA | Diagnostic imaging agent in |
| pendetide | | | | newly-diagnosed prostate |
| | | | | cancer or post-prostatectomy |
| certolizumab | subcutaneous | humanized | TNF | Crohn's disease |
| pegol | | | | |
| cetuximab | intravenous | chimeric | EGFR | Metastatic colorectal carcinoma |
| daclizumab | intravenous | humanized | IL2RA | Prophylaxis of acute organ |
| | | | | rejection in renal transplant |
| daclizumab | subcutaneous | humanized | IL2R | Multiple sclerosis |
| daratumumab | intravenous | fully human | CD38 | Multiple myeloma |
| denosumab | subcutaneous | fully human | RANKL | Postmenopausal women |
| | | | | with osteoporosis |
| dinutuximab | intravenous | chimeric | GD2 | Pediatric high- |
| | | | | risk neuroblastoma |
| dupilumab | subcutaneous | fully human | IL4RA | Atopic dermatitis |
| durvalumab | intravenous | fully human | PD-L1 | Urothelial carcinoma |
| eculizumab | intravenous | humanized | Complement | Paroxysmal nocturnal |
| | | | component 5 | hemoglobinuria |
| elotuzumab | intravenous | humanized | SLAMF7 | Multiple myeloma |
| evolocumab | subcutaneous | fully human | PCSK9 | Heterozygous familial |
| | | | | hypercholesterolemia |
| | | | | Refractory hypercholesterolemia |
| golimumab | subcutaneous | fully human | TNF | Rheumatoid arthritis |
| | | | | Psoriatic arthritis |
| | | | | Ankylosing spondylitis |
| golimumab | intravenous | fully human | TNF | Rheumatoid arthritis |
| ibritumomab | intravenous | murine, | CD20 | Relapsed or refractory low- |
| tiuxetan | | radioimmunotherapy | | grade, follicular, or transformed |
| | | | | B-cell non-Hodgkin's lymphoma |
| idarucizumab | intravenous | humanized Fab | dabigatran | Emergency reversal of |
| | | | | anticoagulant dabigatran |
| infliximab | intravenous | chimeric | TNF alpha | Crohn's disease |
| inflixmab-abda | intravenous | chimeric, biosimilar | TNF | Crohn's disease |
| | | | | Ulcerative colitis |
| | | | | Rheumatoid arthritis |
| | | | | Ankylosing spondylitis |
| | | | | Psoriatic arthritis |
| | | | | Plaque psoriasis |
| infliximab- | intravenous | chimeric, biosimilar | TNF | Crohn's disease |
| dyyb | | | | Ulcerative colitis |
| | | | | Rheumatoid arthritis |
| | | | | Ankylosing spondylitis |
| | | | | Psoriatic arthritis |
| | | | | Plaque psoriasis |
| ipilimumab | intravenous | fully human | CTLA-4 | Metastatic melanoma |
| ixekizumab | subcutaneous | humanized | IL17A | Plaque psoriasis |
| mepolizumab | subcutaneous | humanized | IL5 | Severe asthma |
| natalizumab | intravenous | humanized | alpha-4 integrin | Multiple sclerosis |
| necitumumab | intravenous | fully human | EGFR | Metastatic squamous non-small |
| | | | | cell lung carcinoma |
| nivolumab | intravenous | fully human | PD-1 | Metastatic melanoma |
| nivolumab | intravenous | fully human | PD-1 | Metastatic squamous non-small |
| | | | | cell lung carcinoma |
| obiltoxaximab | intravenous | chimeric | Protective | Inhalational anthrax |
| | | | antigen of | |
| | | | the Anthrax | |
| | | | toxin | |
| obinutuzumab | intravenous | humanized | CD20 | Chronic lymphocytic leukemia |
| ocrelizumab | intravenous | humanized | CD20 | Multiple sclerosis |
| ofatumumab | intravenous | fully human | CD20 | Chronic lymphocytic leukemia |
| olaratumab | intravenous | fully human | PDGFRA | Soft tissue sarcoma |
| omalizumab | intravenous | humanized | IgE | Moderate to severe |
| | | | | persistent asthma |
| palivizumab | intramuscular | humanized | F protein | Respiratory syncytial virus |
| | | | of RSV | |
| panitumumab | intravenous | fully human | EGFR | Metastatic colorectal cancer |
| pembrolizumab | intravenous | humanized | PD-1 | Metastatic melanoma |
| pertuzumab | intravenous | humanized | HER2 | Metastatic breast cancer |
| ramucirumab | intravenous | fully human | VEGFR2 | Gastric cancer |
| ranibizumab | intravitreal | humanized | VEGFR1 | Wet age-related macular |
| | injection | | VEGFR2 | degeneration |
| raxibacumab | intravenous | fully human | Protective | Inhalational anthrax |
| | | | antigen | |
| | | | of Bacillus | |
| | | | anthracis | |
| reslizumab | intravenous | humanized | IL5 | Severe asthma |
| rituximab | intravenous | chimeric | CD20 | B-cell non-Hodgkin's lymphoma |
| secukinumab | subcutaneous | fully human | IL17A | Plaque psoriasis |
| siltuximab | intravenous | chimeric | IL6 | Multicentric Castleman's disease |
| tocilizumab | intravenous | humanized | IL6R | Rheumatoid arthritis |
| tocilizumab | intravenous | humanized | IL6R | Rheumatoid arthritis |
| | subcutaneous | | | Polyarticular juvenile idiopathic |
| | | | | arthritis |
| | | | | Systemic juvenile idiopathic |
| | | | | arthritis |
| trastuzumab | intravenous | humanized | HER2 | Metastatic breast cancer |
| ustekinumab | subcutaneous | fully human | IL12 | Plaque psoriasis |
| | | | IL23 | |
| ustekinumab | subcutaneous | fully human | IL12 | Plaque psoriasis |
| | intravenous | | IL23 | Psoriatic arthritis |
| | | | | Crohn's disease |
| vedolizumab | intravenous | humanized | integrin receptor | Ulcerative colitis |
| | | | | Crohn's disease |

Other representative examples of monoclonal antibodies that may be suitable for use in the present invention are listed in Table 2.

TABLE 2
Therapeutic Monoclonal Antibodies

| Name | Type | Source | Target | Use |
|---|---|---|---|---|
| 3F8 | mab | mouse | GD2 ganglioside | neuroblastoma |
| 8H9 | mab | mouse | B7-H3 | neuroblastoma, sarcoma, |
| | | | | metastatic brain cancers |
| Abagovomab | mab | mouse | CA-125 (imitation) | ovarian cancer |
| Abciximab | Fab | chimeric | CD41 (integrin alpha-IIb) | platelet aggregation inhibitor |
| Abituzumab | mab | humanized | CD51 | cancer |
| Abrilumab | mab | human | integrin α4β7 | inflammatory bowel |
| | | | | disease, ulcerative |
| | | | | colitis, Crohn's disease |
| Actoxumab | mab | human | Clostridium difficile | Clostridium difficile colitis |
| Adalimumab | mab | human | TNF-α | Rheumatoid arthritis, Crohn's |
| | | | | Disease, |
| | | | | Plaque Psoriasis, Psoriatic |
| | | | | Arthritis, Ankylosing |
| | | | | Spondylitis, Juvenile |
| | | | | Idiopathic |
| | | | | Arthritis, Hemolytic disease |
| | | | | of the newborn |
| Adecatumumab | mab | human | EpCAM | prostate and breast cancer |
| Aducanumab | mab | human | beta-amyloid | Alzheimer's disease |
| Afelimomab | F(ab′)₂ | mouse | TNF-α | sepsis |
| Afutuzumab | mab | humanized | CD20 | lymphoma |
| Alacizumab pegol | F(ab′)₂ | humanized | VEGFR2 | cancer |
| Alemtuzumab | mab | humanized | CD52 | Multiple sclerosis |
| Alirocumab | mab | human | PCSK9 | hypercholesterolemia |
| Altumomab | mab | mouse | CEA | colorectal cancer (diagnosis) |
| pentetate | | | | |
| Amatuximab | mab | chimeric | mesothelin | cancer |
| Anatumomab | Fab | mouse | TAG-72 | non-small cell lung |
| mafenatox | | | | carcinoma |
| Anetumab | mab | human | MSLN | cancer |
| ravtansine | | | | |
| Anifrolumab | mab | human | interferon α/β receptor | systemic lupus |
| | | | | erythematosus |
| Anrukinzumab | mab | humanized | IL-13 | asthma |
| (=IMA-638) | | | | |
| Arcitumomab | Fab′ | mouse | CEA | gastrointestinal cancers |
| | | | | (diagnosis) |
| Ascrinvacumab | mab | human | activin receptor-like kinase | cancer |
| | | | 1 | |
| Aselizumab | mab | humanized | L-selectin (CD62L) | severely injured patients |
| Atezolizumab | mab | humanized | CD274 | cancer |
| Atlizumab | mab | humanized | IL-6 receptor | rheumatoid arthritis |
| (=tocilizumab) | | | | |
| Atorolimumab | mab | human | Rhesus factor | hemolytic disease of the |
| | | | | newborn |
| Bapineuzumab | mab | humanized | beta amyloid | Alzheimer's disease |
| Basiliximab | mab | chimeric | CD25 (α chain of IL- | prevention of |
| | | | 2 receptor) | organ transplant rejections |
| Bavituximab | mab | chimeric | phosphatidylserine | cancer, viral infections |
| Bectumomab | Fab′ | mouse | CD22 | non-Hodgkin's |
| | | | | lymphoma (detection) |
| Belimumab | mab | human | BAFF | non-Hodgkin lymphoma etc. |
| Benralizumab | mab | humanized | CD125 | asthma |
| Bertilimumab | mab | human | CCL11 (eotaxin-1) | severe allergic disorders |
| Besilesomab | mab | mouse | CEA-related antigen | inflammatory lesions and |
| | | | | metastases (detection) |
| Bevacizumab | mab | humanized | VEGF-A | metastatic |
| | | | | cancer, retinopathy of |
| | | | | prematurity |
| Bezlotoxumab | mab | human | Clostridium difficile | Clostridium difficile colitis |
| Biciromab | Fab′ | mouse | fibrin II, beta chain | thromboembolism |
| | | | | (diagnosis) |
| Bimagrumab | mab | human | ACVR2B | myostatin inhibitor |
| Bivatuzumab | mab | humanized | CD44 v6 | squamous cell carcinoma |
| mertansine | | | | |
| Blinatumomab | BiTE | mouse | CD19 | pre-B ALL (CD 19+) |
| Blosozumab | mab | humanized | SOST | osteoporosis |
| Bococizumab | mab | humanized | neural apoptosis-regulated | dyslipidemia |
| | | | proteinase 1 | |
| Brazikumab | mab | human | IL23 | Crohn's disease |
| Brentuximab | mab | chimeric | CD30 (TNFRSF8) | hematologic cancers |
| vedotin | | | | |
| Briakinumab | mab | human | IL-12, IL-23 | psoriasis, rheumatoid |
| | | | | arthritis, inflammatory bowel |
| | | | | diseases, multiple sclerosis |
| Brodalumab | mab | human | IL-17 | inflammatory diseases |
| Brolucizumab | mab | humanized | VEGFA | wet age-related macular |
| | | | | degeneration |
| Brontictuzumab | mab | humanized | Notch 1 | cancer |
| Burosumab | mab | human | FGF23 | X-linked hypophosphatemia |
| Cantuzumab | mab | humanized | mucin CanAg | colorectal cancer etc. |
| mertansine | | | | |
| Cantuzumab | mab | humanized | MUC1 | cancers |
| ravtansine | | | | |
| Caplacizumab | mab | humanized | VWF | thrombotic |
| | | | | thrombocytopenic |
| | | | | purpura, thrombosis |
| Capromab | mab | mouse | prostatic carcinoma cells | prostate cancer (detection) |
| pendetide | | | | |
| Carlumab | mab | human | MCP-1 | oncology/immune |
| | | | | indications |
| Catumaxomab | 3funct | rat/mouse hybrid | EpCAM, CD3 | ovarian cancer, |
| | | | | malignant ascites, gastric |
| | | | | cancer |
| cBR96-doxorubicin | mab | humanized | Lewis-Y antigen | cancer |
| immunoconjugate | | | | |
| Cedelizumab | mab | humanized | CD4 | prevention of organ |
| | | | | transplant rejections, |
| | | | | treatment of autoimmune |
| | | | | diseases |
| Certolizumab pegol | Fab′ | humanized | TNF-α | Crohn's disease Rheumatoid |
| | | | | arthritis axial |
| | | | | spondyloarthritis psoriasis |
| | | | | arthritis |
| Cetuximab | mab | chimeric | EGFR | metastatic colorectal |
| | | | | cancer and head and neck |
| | | | | cancer |
| Ch.14.18 | mab | chimeric | GD2 ganglioside | neuroblastoma |
| Citatuzumab | Fab | humanized | EpCAM | ovarian cancer and other |
| bogatox | | | | solid tumors |
| Cixutumumab | mab | human | IGF-1 receptor (CD221) | solid tumors |
| Clazakizumab | mab | humanized | Oryctolagus cuniculus | rheumatoid arthritis |
| Clenoliximab | mab | chimeric | CD4 | rheumatoid arthritis |
| Clivatuzumab | mab | humanized | MUC1 | pancreatic cancer |
| tetraxetan | | | | |
| Codrituzumab | mab | humanized | glypican 3 | cancer |
| Coltuximab | mab | chimeric | CD19 | cancer |
| ravtansine | | | | |
| Conatumumab | mab | human | TRAIL-R2 | cancer |
| Concizumab | mab | humanized | TFPI | bleeding |
| CR6261 | mab | human | Influenza A hemagglutinin | infectious disease/influenza A |
| Crenezumab | mab | humanized | 1-40-β-amyloid | Alzheimer's disease |
| Crotedumab | mab | human | GCGR | diabetes |
| Dacetuzumab | mab | humanized | CD40 | hematologic cancers |
| Daclizumab | mab | humanized | CD25 (α chain of IL- | prevention of organ |
| | | | 2 receptor) | transplant rejections |
| Dalotuzumab | mab | humanized | IGF-1 receptor (CD221) | cancer etc. |
| Daratumumab | mab | human | CD38 (cyclic ADP ribose | cancer |
| | | | hydrolase) | |
| Demcizumab | mab | humanized | DLL4 | cancer |
| Denintuzumab | mab | humanized | CD19 | cancer |
| mafodotin | | | | |
| Denosumab | mab | human | RANKL | osteoporosis, bone |
| | | | | metastases etc. |
| Depatuxizumab | mab | chimeric/humanized | EGFR | cancer |
| mafodotin | | | | |
| Derlotuximab biotin | mab | chimeric | histone complex | recurrent glioblastoma |
| | | | | multiform |
| Detumomab | mab | mouse | B-lymphoma cell | lymphoma |
| Dinutuximab | mab | chimeric | GD2 ganglioside | neuroblastoma |
| Diridavumab | mab | human | hemagglutinin | influenza A |
| Domagrozumab | mab | humanized | GDF-8 | Duchenne muscular |
| | | | | dystrophy |
| Drozitumab | mab | human | DR5 | cancer etc. |
| Duligotumab | mab | human | ERBB3 (HER3) | testicular cancer |
| Dupilumab | mab | human | IL4 | atopic diseases |
| Durvalumab | mab | human | CD274 | cancer |
| Dusigitumab | mab | human | ILGF2 | cancer |
| Ecromeximab | mab | chimeric | GD3 ganglioside | malignant melanoma |
| Eculizumab | mab | humanized | C5 | paroxysmal nocturnal |
| | | | | hemoglobinuria, atypical |
| | | | | HUS |
| Edobacomab | mab | mouse | endotoxin | sepsis caused by Gram- |
| | | | | negative bacteria |
| Edrecolomab | mab | mouse | EpCAM | colorectal carcinoma |
| Efalizumab | mab | humanized | LFA-1 (CD11a) | psoriasis (blocks T- |
| | | | | cell migration) |
| Efungumab | scFv | human | Hsp90 | invasive Candida infection |
| Eldelumab | mab | human | interferon gamma-induced | Crohn's disease, ulcerative |
| | | | protein | colitis |
| Elgemtumab | mab | human | ERBB3 (HER3) | cancer |
| Elotuzumab | mab | humanized | SLAMF7 | multiple myeloma |
| Emactuzumab | mab | humanized | CSF1R | cancer |
| Emibetuzumab | mab | humanized | HHGFR | cancer |
| Emicizumab | mab | humanized | activated F9, F10 | haemophilia A |
| Enavatuzumab | mab | humanized | TWEAK receptor | cancer etc. |
| Enfortumab vedotin | mab | human | AGS-22M6 | cancer expressing Nectin-4 |
| Enoblituzumab | mab | humanized | CD276 | cancer |
| Enokizumab | mab | humanized | IL9 | asthma |
| Ensituximab | mab | chimeric | 5AC | cancer |
| Epratuzumab | mab | humanized | CD22 | cancer, SLE |
| Erenumab | mab | human | CGRP | migraine |
| Erlizumab | F(ab′)₂ | humanized | ITGB2 (CD18) | heart |
| | | | | attack, stroke, traumatic |
| | | | | shock |
| Ertumaxomab | 3funct | rat/mouse hybrid | HER2/neu, CD3 | breast cancer etc. |
| Etaracizumab | mab | humanized | integrin αᵥβ₃ | melanoma, prostate |
| | | | | cancer, ovarian cancer etc. |
| Etrolizumab | mab | humanized | integrin α₇ β₇ | inflammatory bowel disease |
| Evinacumab | mab | human | angiopoietin 3 | dyslipidemia |
| Evolocumab | mab | human | PCSK9 | hypercholesterolemia |
| Exbivirumab | mab | human | hepatitis B surface antigen | hepatitis B |
| Fanolesomab | mab | mouse | CD15 | appendicitis (diagnosis) |
| Farletuzumab | mab | humanized | folate receptor 1 | ovarian cancer |
| Fasinumab | mab | human | HNGF | acute sciatic pain |
| FBTA05 | 3funct | rat/mouse hybrid | CD20 | chronic lymphocytic |
| | | | | leukaemia |
| Felvizumab | mab | humanized | respiratory syncytial virus | respiratory syncytial virus |
| | | | | infection |
| Fezakinumab | mab | human | IL-22 | rheumatoid |
| | | | | arthritis, psoriasis |
| Ficlatuzumab | mab | humanized | HGF | cancer etc. |
| Figitumumab | mab | human | IGF-1 receptor (CD221) | adrenocortical |
| | | | | carcinoma, non-small cell |
| | | | | lung carcinoma etc. |
| Flanvotumab | mab | human | TYRP1(glycoprotein 75) | melanoma |
| Fletikumab | mab | human | IL 20 | rheumatoid arthritis |
| Fontolizumab | mab | humanized | IFN-γ | Crohn's disease etc. |
| Foravirumab | mab | human | rabies virus glycoprotein | rabies (prophylaxis) |
| Fresolimumab | mab | human | TGF-β | idiopathic pulmonary |
| | | | | fibrosis, focal segmental |
| | | | | glomerulosclerosis, cancer |
| Fulranumab | mab | human | NGF | pain |
| Futuximab | mab | chimeric | EGFR | cancer |
| Galcanezumab | mab | humanized | calcitonin | migraine |
| Galiximab | mab | chimeric | CD80 | B-cell lymphoma |
| Ganitumab | mab | human | IGF-1 receptor (CD221) | cancer |
| Gantenerumab | mab | human | beta amyloid | Alzheimer's disease |
| Gavilimomab | mab | mouse | CD147 (basigin) | graft versus host disease |
| Gemtuzumab | mab | humanized | CD33 | acute myelogenous leukemia |
| ozogamicin | | | | |
| Gevokizumab | mab | humanized | IL-1β | diabetes etc. |
| Girentuximab | mab | chimeric | carbonic anhydrase 9 (CA- | clear cell renal cell |
| | | | IX) | carcinoma |
| Glembatumumab | mab | human | GPNMB | melanoma, breast cancer |
| vedotin | | | | |
| Golimumab | mab | human | TNF-α | rheumatoid arthritis, |
| | | | | psoriatic arthritis, ankylosing |
| | | | | spondylitis |
| Gomiliximab | mab | chimeric | CD23 (IgE receptor) | allergic asthma |
| Guselkumab | mab | human | IL23 | psoriasis |
| Ibalizumab | mab | humanized | CD4 | HIV infection |
| Ibritumomab | mab | mouse | CD20 | non-Hodgkin's lymphoma |
| tiuxetan | | | | |
| Icrucumab | mab | human | VEGFR-1 | cancer etc. |
| Idarucizumab | mab | humanized | dabigatran | reversal of anticoagulant |
| | | | | effects of dabigatran |
| Igovomab | F(ab′)₂ | mouse | CA-125 | ovarian cancer (diagnosis) |
| IMAB362 | mab | human | CLDN18.2 | gastrointestinal |
| | | | | adenocarcinomas and |
| | | | | pancreatic tumor |
| Imalumab | mab | human | MIF | cancer |
| Imciromab | mab | mouse | cardiac myosin | cardiac imaging |
| Imgatuzumab | mab | humanized | EGFR | cancer |
| Inclacumab | mab | human | selectin P | cardiovascular disease |
| Indatuximab | mab | chimeric | SDC1 | cancer |
| ravtansine | | | | |
| Indusatumab | mab | human | GUCY2C | cancer |
| vedotin | | | | |
| Inebilizumab | mab | humanized | CD19 | cancer, systemic |
| | | | | sclerosis, multiple sclerosis |
| Infliximab | mab | chimeric | TNF-α | rheumatoid arthritis, |
| | | | | ankylosing spondylitis, |
| | | | | psoriatic arthritis, psoriasis, |
| | | | | Crohn's disease, ulcerative |
| | | | | colitis |
| Inolimomab | mab | mouse | CD25 (α chain of IL- | graft versus host disease |
| | | | 2 receptor) | |
| Inotuzumab | mab | humanized | CD22 | ALL |
| ozogamicin | | | | |
| Intetumumab | mab | human | CD51 | solid tumors (prostate cancer, |
| | | | | melanoma) |
| Ipilimumab | mab | human | CD152 | melanoma |
| Iratumumab | mab | human | CD30 (TNFRSF8) | Hodgkin's lymphoma |
| Isatuximab | mab | chimeric | CD38 | cancer |
| Ixekizumab | mab | humanized | IL 17A | autoimmune diseases |
| Keliximab | mab | chimeric | CD4 | chronic asthma |
| Labetuzumab | mab | humanized | CEA | colorectal cancer |
| Lampalizumab | mab | humanized | CFD | geographic atrophy |
| | | | | secondary to age-related |
| | | | | macular degeneration |
| Lanadelumab | mab | human | kallikrein | angioedema |
| Landogrozumab | mab | humanized | GDF-8 | muscle wasting disorders |
| Lebrikizumab | mab | humanized | IL-13 | asthma |
| Lemalesomab | mab | mouse | NCA-90 (granulocyte | diagnostic agent |
| | | | antigen) | |
| Lerdelimumab | mab | human | TGF beta 2 | reduction of scarring |
| | | | | after glaucoma surgery |
| Lexatumumab | mab | human | TRAIL-R2 | cancer |
| Libivirumab | mab | human | hepatitis B surface antigen | hepatitis B |
| Lifastuzumab | mab | humanized | phosphate-sodium co- | cancer |
| vedotin | | | transporter | |
| Ligelizumab | mab | humanized | IGHE | severe asthma and chronic |
| | | | | spontaneous urticaria |
| Lilotomab | mab | mouse | CD37 | cancer |
| satetraxetan | | | | |
| Lintuzumab | mab | humanized | CD33 | cancer |
| Lirilumab | mab | human | KIR2D | solid and hematological |
| | | | | cancers |
| Lodelcizumab | mab | humanized | PCSK9 | hypercholesterolemia |
| Lorvotuzumab | mab | humanized | CD56 | cancer |
| mertansine | | | | |
| Lucatumumab | mab | human | CD40 | multiple myeloma, non- |
| | | | | Hodgkin's |
| | | | | lymphoma, Hodgkin's |
| | | | | lymphoma |
| Lulizumab pegol | mab | humanized | CD28 | autoimmune diseases |
| Lumiliximab | mab | chimeric | CD23 (IgE receptor) | chronic lymphocytic |
| | | | | leukemia |
| Lumretuzumab | mab | humanized | ERBB3 (HER3) | cancer |
| MABp1 | mab | human | IL1A | colorectal cancer |
| Mapatumumab | mab | human | TRAIL-R1 | cancer |
| Margetuximab | mab | humanized | ch4D5 | cancer |
| Matuzumab | mab | humanized | EGFR | colorectal, lung and stomach |
| | | | | cancer |
| Mavrilimumab | mab | human | GMCSF receptor α-chain | rheumatoid arthritis |
| Mepolizumab | mab | humanized | IL-5 | asthma and white blood cell |
| | | | | diseases |
| Metelimumab | mab | human | TGF beta 1 | systemic scleroderma |
| Milatuzumab | mab | humanized | CD74 | multiple myeloma and other |
| | | | | hematological malignancies |
| Mirvetuximab | mab | chimeric | folate receptor alpha | cancer |
| soravtansine | | | | |
| Mitumomab | mab | mouse | GD3 ganglioside | small cell lung carcinoma |
| Mogamulizumab | mab | humanized | CCR4 | cancer |
| Motavizumab | mab | humanized | respiratory syncytial virus | respiratory syncytial virus |
| | | | | (prevention) |
| Moxetumomab | mab | mouse | CD22 | cancer |
| pasudotox | | | | |
| Muromonab-CD3 | mab | mouse | CD3 | prevention of organ |
| | | | | transplant rejections |
| Nacolomab | Fab | mouse | C242 antigen | colorectal cancer |
| tafenatox | | | | |
| Naptumomab | Fab | mouse | 5T4 | non-small cell lung |
| estafenatox | | | | carcinoma, renal cell |
| | | | | carcinoma |
| Narnatumab | mab | human | RON | cancer |
| Natalizumab | mab | humanized | integrin α₄ | multiple sclerosis, Crohn's |
| | | | | disease |
| Nebacumab | mab | human | endotoxin | sepsis |
| Necitumumab | mab | human | EGFR | non-small cell lung |
| | | | | carcinoma |
| Nemolizumab | mab | humanized | IL31RA | eczema |
| Nesvacumab | mab | human | angiopoietin 2 | cancer |
| Nimotuzumab | mab | humanized | EGFR | squamous cell carcinoma, |
| | | | | head and neck |
| | | | | cancer, nasopharyngeal |
| | | | | cancer, glioma |
| Nivolumab | mab | human | PD-1 | cancer |
| Obiltoxaximab | mab | chimeric | Bacillus anthracis anthrax | Bacillus anthracis spores |
| Obinutuzumab | mab | humanized | CD20 | Chronic lymphatic leukemia |
| Ocaratuzumab | mab | humanized | CD20 | cancer |
| Ocrelizumab | mab | humanized | CD20 | rheumatoid arthritis, lupus |
| | | | | erythematosus etc. |
| Odulimomab | mab | mouse | LFA-1 (CD11a) | prevention of organ |
| | | | | transplant rejections, |
| | | | | immunological diseases |
| Ofatumumab | mab | human | CD20 | chronic lymphocytic |
| | | | | leukemia etc. |
| Olaratumab | mab | human | PDGF-R α | cancer |
| Omalizumab | mab | humanized | IgE Fc region | allergic asthma |
| Onartuzumab | mab | humanized | human scatter factor | cancer |
| | | | receptor kinase | |
| Ontuxizumab | mab | chimeric/humanized | TEM1 | cancer |
| Opicinumab | mab | human | LINGO-1 | multiple sclerosis |
| Oportuzumab | scFv | humanized | EpCAM | cancer |
| monatox | | | | |
| Oregovomab | mab | mouse | CA-125 | ovarian cancer |
| Otelixizumab | mab | chimeric/humanized | CD3 | diabetes mellitus type 1 |
| Otlertuzumab | mab | humanized | CD37 | cancer |
| Oxelumab | mab | human | OX-40 | asthma |
| Ozanezumab | mab | humanized | NOGO-A | ALS and multiple sclerosis |
| Ozoralizumab | mab | humanized | TNF-α | inflammation |
| Pagibaximab | mab | chimeric | lipoteichoic acid | sepsis (Staphylococcus) |
| Palivizumab | mab | humanized | F protein of respiratory | respiratory syncytial virus |
| | | | syncytial virus | (prevention) |
| Panitumumab | mab | human | EGFR | colorectal cancer |
| Pankomab | mab | humanized | tumor specific | ovarian cancer |
| | | | glycosylation of MUC1 | |
| Panobacumab | mab | human | Pseudomonas aeruginosa | Pseudomonas |
| | | | | aeruginosa infection |
| Parsatuzumab | mab | human | EGFL7 | cancer |
| Pascolizumab | mab | humanized | IL-4 | asthma |
| Pasotuxizumab | mab | chimeric/humanized | folate hydrolase | cancer |
| Pateclizumab | mab | humanized | LTA | TNF |
| Patritumab | mab | human | ERBB3 (HER3) | cancer |
| Pembrolizumab | mab | humanized | PDCD1 | melanoma and other cancers |
| Perakizumab | mab | humanized | IL 17A | arthritis |
| Pertuzumab | mab | humanized | HER2/neu | cancer |
| Pexelizumab | scFv | humanized | C5 | reduction of side effects |
| | | | | of cardiac surgery |
| Pidilizumab | mab | humanized | PD-1 | cancer and infectious |
| | | | | diseases |
| Pinatuzumab | mab | humanized | CD22 | cancer |
| vedotin | | | | |
| Pintumomab | mab | mouse | adenocarcinoma antigen | adenocarcinoma (imaging) |
| Placulumab | mab | human | human TNF | pain and inflammatory |
| | | | | diseases |
| Plozalizumab | mab | humanized | CCR2 | diabetic |
| | | | | nephropathy and |
| | | | | arteriovenous graft |
| | | | | patency |
| Polatuzumab | mab | humanized | CD79B | cancer |
| vedotin | | | | |
| Ponezumab | mab | humanized | human beta-amyloid | Alzheimer's disease |
| Priliximab | mab | chimeric | CD4 | Crohn's disease, multiple |
| | | | | sclerosis |
| Pritumumab | mab | human | vimentin | brain cancer |
| Quilizumab | mab | humanized | IGHE | asthma |
| Racotumomab | mab | mouse | N-glycolylneuraminic acid | cancer |
| Radretumab | mab | human | fibronectin extra domain-B | cancer |
| Rafivirumab | mab | human | rabies virus glycoprotein | rabies (prophylaxis) |
| Ralpancizumab | mab | humanized | neural apoptosis-regulated | dyslipidemia |
| | | | proteinase 1 | |
| Ramucirumab | mab | human | VEGFR2 | solid tumors |
| Ranibizumab | Fab | humanized | VEGF-A | macular degeneration (wet |
| | | | | form) |
| Raxibacumab | mab | human | anthrax toxin, protective | anthrax (prophylaxis and |
| | | | antigen | treatment) |
| Refanezumab | mab | humanized | myelin-associated | recovery of motor function |
| | | | glycoprotein | after stroke |
| Regavirumab | mab | human | cytomegalovirus glycoprotein | cytomegalovirus infection |
| | | | B | |
| Reslizumab | mab | humanized | IL-5 | inflammations of the |
| | | | | airways, skin and |
| | | | | gastrointestinal tract |
| Rilotumumab | mab | human | HGF | solid tumors |
| Rinucumab | mab | human | platelet-derived growth | neovascular age-related |
| | | | factor receptor beta | macular degeneration |
| Rituximab | mab | chimeric | CD20 | lymphomas, leukemias, some |
| | | | | autoimmune disorders |
| Robatumumab | mab | human | IGF-1 receptor (CD221) | cancer |
| Romosozumab | mab | humanized | sclerostin | osteoporosis |
| Rontalizumab | mab | humanized | IFN-α | systemic lupus |
| | | | | erythematosus |
| Rovelizumab | mab | humanized | CD11, CD18 | haemorrhagic shock etc. |
| Ruplizumab | mab | humanized | CD154 (CD40L) | rheumatic diseases |
| Sacituzumab | mab | humanized | tumor-associated calcium | cancer |
| govitecan | | | signal transducer 2 | |
| Samalizumab | mab | humanized | CD200 | cancer |
| Sarilumab | mab | human | IL6 | rheumatoid |
| | | | | arthritis, ankylosing |
| | | | | spondylitis |
| Satumomab | mab | mouse | TAG-72 | cancer (diagnosis) |
| pendetide | | | | |
| Secukinumab | mab | human | IL 17A | uveitis, rheumatoid |
| | | | | arthritis psoriasis |
| Seribantumab | mab | human | ERBB3 (HER3) | cancer |
| SGN-CD19A | mab | humanized | CD19 | acute lymphoblastic |
| | | | | leukemia and B-cell non- |
| | | | | Hodgkin lymphoma |
| SGN-CD33A | mab | humanized | CD33 | Acute myeloid leukemia |
| Sibrotuzumab | mab | humanized | FAP | cancer |
| Sifalimumab | mab | humanized | IFN-α | SLE, dermatomyositis, poly |
| | | | | myositis |
| Siltuximab | mab | chimeric | IL-6 | cancer |
| Simtuzumab | mab | humanized | LOXL2 | fibrosis |
| Siplizumab | mab | humanized | CD2 | psoriasis, graft-versus-host |
| | | | | disease (prevention) |
| Sirukumab | mab | human | IL-6 | rheumatoid arthritis |
| Sofituzumab | mab | humanized | CA-125 | ovarian cancer |
| vedotin | | | | |
| Solanezumab | mab | humanized | beta amyloid | Alzheimer's disease |
| Stamulumab | mab | human | myostatin | muscular dystrophy |
| Sulesomab | Fab′ | mouse | NCA-90 (granulocyte | osteomyelitis (imaging) |
| | | | antigen) | |
| Suvizumab | mab | humanized | HIV-1 | viral infections |
| Tabalumab | mab | human | BAFF | B-cell cancers |
| Tacatuzumab | mab | humanized | alpha-fetoprotein | cancer |
| tetraxetan | | | | |
| Tadocizumab | Fab | humanized | integrin α_{IIb}β₃ | percutaneous coronary |
| | | | | intervention |
| Talizumab | mab | humanized | IgE | allergic reaction |
| Tanezumab | mab | humanized | NGF | pain |
| Taplitumomab | mab | mouse | CD19 | cancer |
| paptox | | | | |
| Tarextumab | mab | human | Notch receptor | cancer |
| Tefibazumab | mab | humanized | clumping factor A | Staphylococcus |
| | | | | aureus infection |
| Tenatumomab | mab | mouse | tenascin C | cancer |
| Teneliximab | mab | chimeric | CD40 | autoimmune diseases and |
| | | | | prevention of organ |
| | | | | transplant rejection |
| Teplizumab | mab | humanized | CD3 | diabetes mellitus type 1 |
| Teprotumumab | mab | human | IGF-1 receptor (CD221) | hematologic tumors |
| Tetulomab | mab | humanized | CD37 | cancer |
| Tezepelumab | mab | human | TSLP | asthma, atopic dermatitis |
| Ticilimumab | mab | human | CTLA-4 | cancer |
| (=tremelimumab) | | | | |
| Tigatuzumab | mab | humanized | TRAIL-R2 | cancer |
| Tildrakizumab | mab | humanized | IL23 | immunologically mediated |
| | | | | inflammatory disorders |
| Tocilizumab | mab | humanized | IL-6 receptor | rheumatoid arthritis |
| (=atlizumab) | | | | |
| Toralizumab | mab | humanized | CD154 (CD40L) | rheumatoid arthritis, lupus |
| | | | | nephritis etc. |
| Tovetumab | mab | human | CD140a | cancer |
| Tralokinumab | mab | human | IL-13 | asthma etc. |
| Trastuzumab | mab | humanized | HER2/neu | breast cancer |
| Trastuzumab | mab | humanized | HER2/neu | breast cancer |
| emtansine | | | | |
| Tremelimumab | mab | human | CTLA-4 | cancer |
| Trevogrumab | mab | human | growth differentiation | muscle atrophy due to |
| | | | factor 8 | orthopedic disuse |
| | | | | and sarcopenia |
| Tucotuzumab | mab | humanized | EpCAM | cancer |
| celmoleukin | | | | |
| Ublituximab | mab | chimeric | MS4A1 | cancer |
| Ulocuplumab | mab | human | CXCR4 (CD184) | hematologic malignancies |
| Urelumab | mab | human | 4-1BB (CD137) | cancer etc. |
| Urtoxazumab | mab | humanized | Escherichia coli | diarrhea caused by E. coli |
| Ustekinumab | mab | human | IL-12, IL-23 | multiple sclerosis, psoriasis, |
| | | | | psoriatic arthritis |
| Utomilumab | mab | human | 4-1BB (CD137) | cancer |
| Vandortuzumab | mab | humanized | STEAP1 | cancer |
| vedotin | | | | |
| Vantictumab | mab | human | Frizzled receptor | cancer |
| Vanucizumab | mab | humanized | angiopoietin 2 | cancer |
| Varlilumab | mab | human | CD27 | solid tumors and |
| | | | | hematologic malignancies |
| Vedolizumab | mab | humanized | integrin α₄β₇ | Crohn's disease, ulcerative |
| | | | | colitis |
| Veltuzumab | mab | humanized | CD20 | non-Hodgkin's lymphoma |
| Vepalimomab | mab | mouse | AOC3 (VAP-1) | inflammation |
| Vesencumab | mab | human | NRP1 | solid malignancies |
| Visilizumab | mab | humanized | CD3 | Crohn's disease, ulcerative |
| | | | | colitis |
| Vobarilizumab | mab | humanized | IL6R | inflammatory autoimmune |
| | | | | diseases |
| Volociximab | mab | chimeric | integrin α₅β₁ | solid tumors |
| Vorsetuzumab | mab | humanized | CD70 | cancer |
| mafodotin | | | | |
| Votumumab | mab | human | tumor antigen CTAA16.88 | colorectal tumors |
| Zalutumumab | mab | human | EGFR | squamous cell carcinoma of |
| | | | | the head and neck |
| Zanolimumab | mab | human | CD4 | rheumatoid arthritis, |
| | | | | psoriasis, T-cell lymphoma |
| Zatuximab | mab | chimeric | HER1 | cancer |
| Zolimomab aritox | mab | mouse | CD5 | systemic lupus |
| | | | | erythematosus, graft-versus- |
| | | | | host disease |

Representative therapeutic uses (e.g., approved indications and proposed indications) for (and targets of) the monoclonal antibodies are set forth in Tables 1 and 2. In some embodiments, the patient (e.g., human) has been diagnosed with a disease or condition such as Crohn's disease, ulcerative colitis or indeterminant colitis, cancer (e.g., neuroblastoma, multiple myeloma, non-small cell lung cancer, Merkel cell cancer, leukemia, colorectal cancer, sarcoma, lymphoma, breast cancer, gastric cancer and melanoma), transplant rejection, hypercholesterolemia, Clostridium difficle infection, sepsis, osteoporosis, multiple sclerosis, anthrax and asthma.

The co-administration of colchicine and/or hydroxychloroquine with a therapeutic monoclonal antibody may be particularly advantageous in chronically administered monoclonal antibodies, since duration of the treatment with the monoclonal antibodies contributes to the development of anti-drug antibodies. Examples of monoclonal antibodies with long duration of use include those that are indicated for the treatment of autoimmune diseases. In particular, inhibitors of tumor necrosis factor alpha (TNF-α) that are used to treat the inflammatory bowel diseases (ulcerative colitis and Crohn's disease) are commonly prescribed together with thiopurines to prevent antibody formation. These TNF-α inhibitors would include, but are not limited to: adalimumab, certolizumab, golimumab, infliximab and ozoralizumab. Yet other monoclonal antibodies used to treat inflammatory bowel diseases that may be particularly suited for use the present invention include inhibitors of integrin a (e.g., abrilumab, etaracizumab, etrolizumab, natalizumab, vedolizumab, volociximab). Cancer is yet another disease that may entail prolonged treatment with a monoclonal antibody. For example, volociximab is an inhibitor of integrin-α that is used for the treatment of solid tumors. Thus, anti-cancer monoclonal antibodies may also be useful in practice of the present invention.

The terms “co-administering” or “co-administration” as used herein embrace administration of the therapeutically effective amount of the monoclonal antibody and the effective amount of hydroxychloroquine and/or colchicine simultaneously, either in the same or different dosage forms, or at different times, provided that they are made during the treatment “period” which for purposes of the present invention, includes the time while the monoclonal antibody is still present in the blood serum. That is, the monoclonal antibody and hydroxychloroquine and/or colchicine can be administered together or separately, for example, at different times and in different formulations and/or via different routes of administration. In some embodiments, hydroxychloroquine and/or colchicine may be administered to the patient can be prior to initiation of the monoclonal antibody therapy, e.g., to build up levels of hydroxychloroquine in the system to prevent anti-drug antibody formation. In some embodiments, hydroxychloroquine and/or colchicine may be initiated at the time of the monoclonal antibody therapy and may continue for the duration of therapy. In various embodiments, co-administering hydroxychloroquine and/or colchicine to the patient can be after administering the monoclonal antibody.

Suitable dosages for the active compounds such as a monoclonal antibody, colchicine and/or hydroxychloroquine can be those dosages presently used in connection with approved indications. Advantages of the present invention, however, are that these dosages can be lowered and/or administered less frequently due to the combined action of the two agents.

Therapeutically effective amounts of monoclonal antibodies depend upon many factors, including for example, the nature and severity of the disease or condition, the age and general health and weight of the patient. Generally, therapeutically effective dosage amounts are known in the art.

Hydroxychloroquine and colchicine may be administered as a free base or in the form of a pharmaceutically acceptable hydrate, solvate or salt. All such forms are embraced by the terms “hydroxychloroquine” and “colchicine”. Hydroxychloroquine is advantageously administered in the form of a sulfate salt. In some embodiments, administration of hydroxychloroquine and/or colchicine to the patent can include daily administration, or every other day, to the patient during monoclonal antibody therapy of the patient. Administering hydroxychloroquine and/or colchicine can begin days or weeks before beginning monoclonal antibody therapy, or can being contemporaneous with initiating monoclonal antibody therapy, or after such therapy has begun. Hydroxychloroquine and/or colchicine may also be administered every third day, every fourth day, or weekly to the patient.

An “effective amount” of the hydroxychloroquine embraces amounts that result in a clearance time of the monoclonal antibody from the circulation or blood serum of the patient that is increased relative to the same regimen of treatment with the monoclonal antibody but without the co-administration of colchicine and/or hydroxychloroquine. In some embodiments, the daily dose of hydroxychloroquine ranges between about 5 mg and about 800 mg, between about 5 mg and about 600 mg, between about 25 mg and about 600 mg, or between about 100 mg and about 400 mg. In some embodiments, dosing of hydroxychloroquine may include an initial dosing following by a maintenance dosing schedule. Thus, for example, an initial dose (in the form of the sulfate salt) may range from about 25 to about 600 mg (19.4 to 465 mg base), taken orally once daily, or in some other embodiments an initial loading of 800 mg. The initial dose may be administered from one to about twelve weeks. A maintenance dose (in the form of the sulfate salt) may range from about 5 to about 400 mg (3.9 mg to 310 mg base) taken orally once daily. Hydroxychloroquine—sulfate salt may be commercially available (PLAQUENIL) in the form of 200 mg tablets.

In general, the daily dose of colchicine may range from about 0.05 mg to about 5 mg, and in some embodiments from about 0.07 mg to about 3.5 mg, and in some other embodiments, from about 0.08 mg to about 3 mg, and in yet other embodiments from about 0.1 mg to about 2.4 mg.

Those skilled in the art appreciate that the dosage regimen of hydroxychloroquine may be adjusted, depending upon the needs of the patient. For example, the dose may need to be reduced, at least temporarily, if the patient develops any adverse side effects. Then after 5 to 10 days the dose may gradually be increased to a recommended final dose. Hydroxychloroquine is advantageously administered with a solid or liquid meal (e.g., milk).

In terms of duration of a therapy period, treatment with the monoclonal antibody to the patient may include administering the patient in intervals of about one week, about two weeks, about three weeks, about four weeks, about five weeks, about six weeks, about seven weeks, about eight weeks, about nine weeks, about 10 weeks, about 11 weeks, or about 12 weeks. That is, the interval can be about one week to about 12 weeks, including each of the other time intervals disclosed herein. In view of the advantages of the present invention, however, not only can the dosage amounts be decreased, alternatively or in conjunction therewith, the monoclonal antibody can be administered less frequently in comparison to administering the monoclonal antibody to a patient not being administered hydroxychloroquine and/or colchicine. For example, where a monoclonal antibody is administered such as infused in eight week intervals, the combination therapy of the present invention may extend the administration of the monoclonal antibody to 10 week or 12 week intervals.

Co-administration also entails administration of each agent in accordance via routes known to be effective and safe. For example, the present methods may include administering hydroxychloroquine and/or colchicine orally, and in the case of colchicine, orally or parenterally (e.g., intravenously). The present invention may include administering the monoclonal antibody to the patient subcutaneously such as intravenously. By way of illustration, infliximab can be administered intravenously and hydroxychloroquine and/or colchicine can be administered orally. As another example, adalimumab can be administered subcutaneously and hydroxychloroquine and/or colchicine can be administered orally. By way of additional illustration, infliximab can be administered intravenously and colchicine can be administered intravenously. As another example, adalimumab can be administered subcutaneously and colchicine can be administered intravenously.

Therapeutic combinations of the present invention include a therapeutically effective amount of a monoclonal antibody, and an effective amount of colchicine and/or hydroxychloroquine, or a combination thereof. These agents may be formulated in the same or different dosage forms.

Monoclonal antibodies are typically formulated for parenteral (e.g., intravenous, intraperitoneal, infusion, intraarterial, intramuscular, subcutaneous) administration. Colchicine may also be formulated for parenteral administration, which may be advantageous in embodiments wherein the monoclonal antibody is being used as an anti-cancer agent. Pharmaceutically acceptable carriers or vehicles include nontoxic buffers such as phosphate, citrate, and other organic acids; salts such as sodium chloride; antioxidants including ascorbic acid and methionine; preservatives (e.g., octadecyldimethylbenzyl ammonium chloride; hexamethonium chloride; benzalkonium chloride; benzethonium chloride; phenol, butyl or benzyl alcohol; alkyl parabens, such as methyl or propyl paraben; catechol; resorcinol; cyclohexanol; 3-pentanol; and m-cresol); low molecular weight polypeptides (e.g., less than about 10 amino acid residues); proteins such as serum albumin, gelatin, or immunoglobulins; hydrophilic polymers such as polyvinylpyrrolidone; amino acids such as glycine, glutamine, asparagine, histidine, arginine, or lysine; carbohydrates such as monosaccharides, disaccharides, glucose, mannose, or dextrins; chelating agents such as EDTA; sugars such as sucrose, mannitol, trehalose or sorbitol; salt-forming counterions such as sodium; metal complexes (e.g., Zn-protein complexes); and non-ionic surfactants such as TWEEN or polyethylene glycol (PEG). More particularly, preparations for parenteral administration include sterile aqueous or non-aqueous solutions, suspensions, and emulsions. Examples of non-aqueous solvents include propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media, such as 0.01-0.1M and preferably 0.05M phosphate buffer or 0.8% saline. Other common parenteral carriers or vehicles include sodium phosphate solutions, Ringer's dextrose, dextrose and sodium chloride, lactated Ringer's, and fixed oils. Intravenous vehicles include fluid and nutrient replenishers, electrolyte replenishers, such as those based on Ringer's dextrose, and the like. Preservatives and other additives may also be present such as for example, antimicrobials, antioxidants, chelating agents, and inert gases and the like.

The composition should be sterile and fluid for purposes of ease of syringability. Sterile injectable solutions can be prepared by incorporating the monoclonal antibody and the vehicle, in the required amount followed by filtered sterilization. Generally, dispersions are prepared by incorporating the monoclonal antibody into a sterile vehicle including a basic dispersion medium. In the case of sterile powders for the preparation of sterile injectable solutions, one method of preparation is vacuum drying and freeze-drying, which yields a powder of the monoclonal antibody from a previously sterile-filtered solution thereof. The preparations for injections are processed, filled into containers such as ampoules, bags, bottles, syringes or vials, and sealed under aseptic conditions according to methods known in the art.

As may be appropriate, other formulation types and routes of administration (e.g., topical, transdermal, oral, rectal, pulmonary) may be appropriate, depending for example on the monoclonal antibody and the indication being treated.

In some embodiments, hydroxychloroquine and/or colchicine is administered orally, optionally in combination with one of more conventional pharmaceutically acceptable carriers and/or excipients. Oral formulations containing hydroxychloroquine and/or colchicine may include tablets, capsules, buccal forms, troches, lozenges and oral liquids such as suspensions and solutions. Capsules can contain mixtures of active compound(s) with inert filler(s) and/or diluent(s) such as the pharmaceutically acceptable starches (e.g., corn, potato or tapioca starch), sugars, artificial sweetening agents, powdered celluloses), flours, gelatins, gums, and the like. Tablet formulations can be made by conventional compression, wet granulation or dry granulation methods and utilize pharmaceutically acceptable diluents, binding agents, lubricants, disintegrants, surface modifying agents (including surfactants), suspending or stabilizing agents, including magnesium stearate, stearic acid, sodium lauryl sulfate, talc, sugars, lactose, dextrin, starch, gelatin, cellulose, methyl cellulose, microcrystalline cellulose, sodium carboxymethyl cellulose, carboxymethyl cellulose calcium, polyvinylpyrrolidine, alginic acid, acacia gum, xanthan gum, sodium citrate, complex silicates, calcium carbonate, glycine, sucrose, sorbitol, dicalcium phosphate, calcium sulfate, lactose, kaolin, mannitol, sodium chloride, low melting waxes, and ion exchange resins. Preferred surface modifying agents include nonionic and anionic surface modifying agents. Representative examples of surface modifying agents include poloxamer 188, benzalkonium chloride, calcium stearate, cetostearl alcohol, cetomacrogel emulsifying wax, sorbitan esters, colloidal silicon dioxide, phosphates, sodium dodecylsulfate, magnesium aluminum silicate, and triethanolamine. Oral formulations herein can utilize standard delay or time release formulations to alter the absorption of the hydroxychloroquine.

In some embodiments, hydroxychloroquine may be formulated in the form of a tablet, along with pharmaceutically acceptable carriers and excipients, including dibasic calcium phosphate, hydroxypropyl methylcellulose, magnesium stearate, polyethylene glycol 400, Polysorbate 80, starch and titanium dioxide. In some embodiments, colchicine is administered in the form of a tablet.

Pharmaceutically acceptable liquid carriers include water, organic solvents, mixtures of both, and pharmaceutically acceptable oils or fats. The compositions may also contain one or more pharmaceutically acceptable excipients or additives such as solubilizers, emulsifiers, buffers, preservatives, sweeteners, flavoring agents, suspending agents, thickening agents, colors, viscosity regulators, stabilizers, and osmo-regulators.

Kits of the present invention include the therapeutic combination, which in turn includes a therapeutically effective amount of a monoclonal antibody, and an effective amount of colchicine or hydroxychloroquine, or a combination thereof. The kit may include both agents in a single dosage form or in separate dosage forms, in which case the respective dosage forms may be disposed in separate containers in the kit. The kit may further include a label or insert that includes printed instructions for using the therapeutic combination to practice the methods described herein. The instructions may be those customarily used in commercial packages of therapeutic products and may contain information about indications, usage, dosage, administration, contraindications and/or warnings concerning use of the products, etc.

Suitable containers include, for example, bottles, vials, syringes, blister pack, and the like. The container can be formed from a variety of materials such as glass or plastic. The container can hold a monoclonal antibody and the like or a formulation thereof which is effective, for treating the condition and may have a sterile access port (e.g., the container can be an intravenous solution bag or a vial having a stopper pierceable by a hypodermic injection needle). The kit may further include another container including a pharmaceutically acceptable buffer, such as bacteriostatic water for injection (BWFI), phosphate-buffered saline, Ringer's solution and dextrose solution. A kit can further include other materials desirable from a commercial and user standpoint, including other buffers, diluents, filters, needles, and syringes. Thus, in some embodiments, a kit may include a first container with a monoclonal antibody contained therein, a second container with a hydroxychloroquine and/or colchicine contained therein, and optionally, a third container including a pharmaceutically-acceptable buffer, such as bacteriostatic water for injection (BWFI), phosphate-buffered saline, Ringer's solution and dextrose solution.

In some embodiments, the kits are customized for the delivery of solid oral forms of hydroxychloroquine and/or colchicine, such as by tablets or capsules. Such a kit can include a number of unit dosages, such as a card having the dosages oriented in the order of their intended use. An example of such a kit is a “blister pack”. Blister packs are well known in the package industry and are widely used for packaging pharmaceutical unit dosage forms. If desired, a memory aid can be provided, for example, in the form of numbers, letters, or other markings or with a calendar insert, designating the days in the treatment schedule in which the dosages can be administered. In other embodiments, the kits are customized for the delivery of a parenteral delivery of colchicine, which may be included in the same dosage formulation as the monoclonal antibody, or a different dosage formulation.

In some embodiments, the kit may include a container for containing the separate pharmaceutical compositions such as a divided bottle or a divided foil packet; however, the separate pharmaceutical compositions can be contained with a single, undivided container. Typically, the kit includes descriptions for the co-administration of the separate compositions. Kits of the present invention may be particularly advantageous when the separate compositions are administered in different dosage forms (e.g., hydroxychloroquine and/or orally and a monoclonal antibody parenterally), and/or are administered at different dosage intervals, and/or when titration of the individual components of the therapeutic combination is desired by the prescribing physician.

The present invention may also have utility in diagnostic applications, where the monoclonal antibody is being used to detect the presence or severity of a disease or other pathological condition. Thus, further aspects of the present invention may include a method for enhancing the efficacy of monoclonal antibody diagnosis, which entails co-administering a diagnostically effective amount of a monoclonal antibody, or a functional fragment thereof, which is optionally labeled (e.g., with a radio-label or a fluorescent label), and an effective amount of colchicine or hydroxychloroquine, or a combination thereof, to a patient in need thereof. A related aspect is directed to a method of increasing the time a diagnostic monoclonal antibody remains in the circulation of a patient, which entails co-administering an effective amount of the diagnostic monoclonal antibody which is optionally labeled, or a functional fragment thereof, and an effective amount of colchicine or hydroxychloroquine, or a combination thereof, to a patient in need thereof, wherein the time the monoclonal antibody remains in the circulation (e.g., blood serum) of the patient is increased relative to the same regimen of administration of the diagnostic monoclonal antibody but without the co-administration of effective amount of the colchicine and/or hydroxychloroquine. Yet another aspect of the present invention is directed to a diagnostic combination, which includes a diagnostically effective amount of an optionally labeled monoclonal antibody, and an effective amount of colchicine or hydroxychloroquine, or a combination thereof. A further aspect of the present invention is directed to a kit, which includes a diagnostic combination, which includes a diagnostically effective amount of an optionally labeled monoclonal antibody, and an effective amount of colchicine or hydroxychloroquine, or a combination thereof. The kit may include both agents in a single dosage form or in separate dosage forms, in which case the respective dosage forms may be disposed in separate containers in the kit. The kit may further include printed instructions for using the diagnostic combination to practice the methods described herein. Diagnostic labels and amounts of antibodies for use in diagnostic methods are known in the art.

All publications cited in the specification, including patent publications and non-patent publications, are indicative of the level of skill of those skilled in the art to which this invention pertains. All these publications are herein incorporated by reference to the same extent as if each individual publication were specifically and individually indicated as being incorporated by reference.

Although the invention described herein has been described with reference to particular embodiments, it is to be understood that these embodiments are merely illustrative of the principle and applications described herein. It is therefore to be understood that numerous modifications may be made to the illustrative embodiments and that other arrangements may be devised without departing from the spirit and scope of the various embodiments described herein as defined by the amended claims.

What is claimed is:

1. A method for enhancing the efficacy of monoclonal antibody therapy, which entails co-administering a therapeutic monoclonal antibody, or a functional fragment thereof, and an effective amount of colchicine or hydroxychloroquine, or a combination thereof, to a patient in need thereof.

2. A method of increasing time that a monoclonal antibody remains in the circulation of a patient, which entails co-administering a therapeutic monoclonal antibody, or a functional fragment of the monoclonal antibody, and an effective amount of colchicine or hydroxychloroquine, or a combination thereof, to a patient in need thereof, wherein the time the monoclonal antibody remains in the circulation of the patient is increased relative to the same regimen of administration of the monoclonal antibody but without the effective amount co-administration of colchicine and/or hydroxychloroquine.

3. The method of claim 1, wherein the monoclonal antibody is administered parenterally.

4. The method of claim 3, wherein the monoclonal antibody is administered intravenously.

5. The method of claim 3, wherein the monoclonal antibody is administered subcutaneously.

6. The method of claim 1, wherein the monoclonal antibody is a human monoclonal antibody.

7. The method of claim 1, wherein the monoclonal antibody is a humanized monoclonal antibody.

8. The method of claim 1, wherein the monoclonal antibody is a chimeric monoclonal antibody.

9. The method of claim 1, wherein the monoclonal antibody is a murine monoclonal antibody.

10. The method of claim 1, wherein the monoclonal antibody is conjugated.

11. The method of claim 10, wherein the monoclonal antibody is conjugated to a drug.

12. The method of claim 1, wherein the monoclonal antibody inhibits tissue necrosis factor—alpha (TNF-α).

13. The method of claim 12, wherein the monoclonal antibody is selected from the group consisting of adalimumab, certolizumab pegol, golimumab, and infliximab.

14. The method of claim 13, wherein the monoclonal antibody is infliximab.

15. The method of claim 1, wherein the hydroxychloroquine and/or colchicine is administered orally.

16. The method of claim 1, wherein the hydroxychloroquine is administered in the form of a sulfate salt.

17. The method of claim 16, wherein the hydroxychloroquine is administered in an amount of about 5 mg to about 800 mg.

18. The method of claim 1, wherein the monoclonal antibody is an inhibitor of integrin-α.

19. The method of claim 1, wherein colchicine is administered parenterally.

20. A therapeutic combination, comprising a therapeutically effective amount of a monoclonal antibody, or a functional fragment thereof, and an effective amount of colchicine or hydroxychloroquine, or a combination thereof.

21. A kit, comprising a therapeutic combination comprising a therapeutically effective amount of a monoclonal antibody, or a functional fragment thereof, and an effective amount of colchicine or hydroxychloroquine, or a combination thereof.

22. The kit of claim 21, comprising a first container comprising the monoclonal antibody in a formulation suitable for parental administration, and a second container comprising the hydroxychloroquine, colchicine or a combination thereof, in a dosage form suitable for oral administration.

23. The kit of claim 21, comprising a first container comprising the monoclonal antibody in a formulation suitable for parental administration, and a second container comprising the colchicine in a dosage form suitable for parenteral administration.
